(19) United States
(12) Patent Application Publication
(10) Pub. No.: US 2025/0092432 A1
(43) Pub. Date: Mar. 20, 2025

(54) NITRILASE MUTANT AND USE THEREOF IN CATALYTIC SYNTHESIS OF 2-CHLORONICOTINIC ACID

(71) Applicant: ZHEJIANG UNIVERSITY OF TECHNOLOGY, HANGZHOU, ZHEJIANG PROVINCE (CN)

(72) Inventors: RENCHAO ZHENG, HANGZHOU, ZHEJIANG PROVINCE (CN); ANDI DAI, HANGZHOU, ZHEJIANG PROVINCE (CN); ZHEMING WU, HANGZHOU, ZHEJIANG PROVINCE (CN); YUGUO ZHENG, HANGZHOU, ZHEJIANG PROVINCE (CN)

(21) Appl. No.: 18/018,561

(22) PCT Filed: Apr. 19, 2021
(86) PCT No.: PCT/CN2021/088232

§ 371 Date: Jan. 28, 2023

(30) Foreign Application Priority Data
Oct. 9, 2020 (CN) 202011074098.7

(51) Int. Cl. C12P 17/12; C12N 9/78; C12P 7/42

(52) U.S. Cl. CPC C12P 17/12; C12N 9/78; C12P 7/42; C12Y 305/05001

(57) ABSTRACT

The present invention discloses a nitrilase mutant and application thereof in catalytic synthesis of 2-chloronicotinic acid, and belongs to the technical field of enzyme engineering. The nitrilase mutant has an amino acid sequence shown in SEQ ID NO.4, that is, 167th tryptophan W of a parent nitrilase is mutated into glycine G. According to the nitrilase mutant provided by the present invention, the hydration activity of the parent nitrilase to 2-chloronicotinonitrile is eliminated, a byproduct of 2-chloronicotinamide is not generated in the catalytic process, the nitrile hydrolysis activity is greatly improved, and 2-chloronicotinonitrile can be specifically subjected to catalytic hydrolysis to synthesize 2-chloronicotinic acid. Therefore, the nitrilase mutant has an important potential in enzymatic industrial synthesis of 2-chloronicotinic acid.

This is a U.S. national stage application of PCT Application No. PCT/CN2021/088232 under 35 U.S.C. 371, filed Apr. 19, 2021 in Chinese, claiming priority of Chinese Application No. 202011074098.7, filed Oct. 9, 2020, all of which are hereby incorporated by reference.

### FIELD OF TECHNOLOGY

The present invention relates to the technical field of enzyme engineering and mainly relates to a nitrilase mutant and application thereof in catalytic synthesis of 2-chloronicotinic acid.

### BACKGROUND TECHNOLOGY

2-chloronicotinic acid is an important nitrogen heterocyclic fine chemical intermediate and widely used for synthesis of pesticides and pharmaceutical chemicals. In the field of pesticides, 2-chloronicotinic acid can be used for synthesizing a sulfonylurea herbicide nicosulfuron, an amide herbicide diflufenican, an amide fungicide boscalid and a triazolethione series fungicide active compound. In the field of medicines, 2-chloronicotinic acid can be used for synthesizing antibiotics and medicines for treating cardiovascular diseases, such as an anti-AIDS medicine Nevirapine, an antidepressant mirtazapine, an anti-inflammatory medicine pranoprofen, an anti-inflammatory analgesics niflumic acid, mefenamic acid and the like. Since 2-chloronicotinic acid is widely used in the pesticides and medicines, its market demand is increasing.

At present, 2-chloronicotinic acid is mainly produced by a chemical method including nicotinic acid nitrogen oxidation-chlorination-hydrolysis method, 3-cyanopyridine oxidation-chlorination-hydrolysis method, 3-methylpyridine chlorination-oxidation method, andalkenyl ether and ethyl cyanoacetate ring-forming method, etc. However, these methods have the disadvantages of severe reaction conditions, environmental pollution, high equipment requirements, and the like. Therefore, the development of a clean and efficient production technology for 2-chloronicotinic acid has an important industrial application value.

In recent years, breakthrough advances in the field of biotechnology have made biocatalysis play an increasingly important role in the chemical industry. The biocatalysis gradually becomes one of the most promising technologies for industrial sustainability. The biocatalysis of a nitrile compound for hydrolysis and hydration has the obvious advantages of high-efficient process, environmental friendliness, high chemoselectivity, regioselectivity, and stereoselectivity, and the like, becomes an important method for industrial synthesis of carboxylic acid and amide, and successfully realizes industrial synthesis of bulk and fine chemicals such as acrylamide, nicotinamide, and the like.

Nitrilase is capable of catalyzing the hydrolysis of a nitrile compound to the corresponding carboxylic acid and ammonia. However, so far, no nitrilase has been reported to be capable of hydrolyzing 2-chloronicotinonitrile into 2-chloronicotinic acid. Researches show that some nitrilases have a nitrile hydration activity at the same time and catalyze a nitrile compound to generate a corresponding amide. The nitrilase has the catalytic properties of both nitrile hydrolysis and nitrile hydration activity, which provides a new visual angle for the development of new functions of biological organic synthesis.

The molecular modification of protein is an effective means for regulating and controlling the catalytic properties of nitrilase, such that the problem needed to be solved by a person skill in the art is to develop a mutant capable of specifically hydrolyzing 2-chloronicotinonitrile to synthesize 2-chloronicotinic acid through the molecular modification and used for the biological organic synthesis of 2-chloronicotinic acid.

### SUMMARY OF INVENTION

The object of the present invention is to modify a nitrilase from Rhodococcus zopfii by a protein engineering technology and to construct a mutant capable of specifically hydrolyzing 2-chloronicotinonitrile to synthesize 2-chloronicotinic acid. The mutant is of great significance for realizing efficient and green production of 2-chloronicotinic acid.

To achieve the above objective, the present invention uses the following technical solutions.

The present invention randomly mutates a coding gene (SEQ ID NO.1) of nitrilase from R. zopfii by using an error-prone PCR technology, specifically, firstly, a T7 primer is used for PCR amplification, mutation is randomly introduced to obtain a nucleotide sequence of a nitrilase mutant, the nucleotide sequence is ligated to an expression vector pET-28b (+), the vector is introduced into a host Escherichia coli, after induced expression, and a mutant with reduced nitrile hydration activity and improved nitrile hydrolysis activity is obtained through high-throughput screening. It is found by a sequencing analysis that a Try167 in the nitrilase from R. zopfii shown in SEQ ID NO.2 is mutated. A site-directed mutagenesis is combined to perform site-directed mutagenesis at position 167 to obtain a mutant with eliminated nitrile hydration activity and further improved nitrile hydrolysis activity, that is, a nitrilase mutant W167G capable of efficiently catalyzing 2-chloronicotinonitrile to synthesize 2-chloronicotinic acid.

The present invention provides a nitrilase mutant having an amino acid sequence shown in SEQ ID NO.4, that is, Try 167 of a parent nitrilase having an amino acid sequence shown in SEQ ID NO.2 is mutated into glycine G.

The present invention further provides a gene encoding the nitrilase mutant, wherein a nucleotide sequence of the gene is shown in SEQ ID No.3.

The present invention further provides a recombinant plasmid containing the gene. Preferably, the original vector is pET-28b (+).

The present invention further provides a recombinant engineered bacterium comprising the recombinant plasmid. The recombinant plasmid is transformed into a host cell to obtain the recombinant engineered bacterium. The host cell can be a variety of conventional host cells in the art. Preferably, the host cell is Escherichia coli BL21 (DE3).

Another object of the present invention is to provide application of the nitrilase mutant in catalytic hydrolysis of 2-chloronicotinonitrile to synthesize 2-chloronicotinic acid.

The application comprises subjecting a recombinant engineered bacterium comprising a gene encoding the nitrilase mutant to induced expression to obtain a wet bacterial cell, subjecting an immobilized cell of the wet bacterial cell or the wet bacterial cell to ultrasonication, extracting a pure enzyme as a catalyst, forming a reaction system by using 2-chloronicotinonitrile as a substrate and a NaH₂PO₄-Na₂HPO₄ buffer solution with a pH value of 6-8 as a reaction medium, performing reaction at 25-45° C., and performing separation and purification to obtain 2-chloronicotinic acid after the reaction.

Preferably, in the reaction system, the amount of the catalyst is 0.2-3 g/L based on the dry weight of the bacterial cell, and the concentration of the substrate is 50-500 mM.

More preferably, the bacterial cell collected after the induced culture is used as the catalyst, the NaH₂PO₄-Na₂HPO₄ buffer solution with a concentration of 200 mM and a pH value of 7 is used as the reaction medium, in the reaction system, the concentration of the substrate 2-chloronicotinonitrile is 300 mM, the bacterial cell is 2 g/L (by dry weight), and the reaction is performed at 30° C. for 30-40 hours.

A preparation method of the wet bacterial cell is as follows: inoculating an engineered bacterium containing a nitrilase mutant gene into an LB liquid medium containing 50 μg/mL of kanamycin, culturing the bacterium at 37° C. and 200 rpm overnight, transferring the bacterium to a fresh LB liquid medium containing 50 μg/mL of kanamycin with an inoculum size of 2% (volume concentration), culturing the bacterium at 37° C. and 180 rpm until a concentration OD₆₀₀ of the bacterial cell is 0.4-0.8, adding IPTG with a final concentration of 0.1-1 mM to the medium, and performing induced culture at 28° C. and 180 rpm for 12 hours. A fermentation broth is centrifuged at 4° C. and 8,000 rpm for 10 min and the bacterial cell is collected. The LB liquid medium consists of 10 g/L of peptone, 5 g/L of a yeast extract, and 10 g/L of NaCl, with a pH of 7.0.

The present invention has the following beneficial effects:

According to the nitrilase mutant W167G provided by the present invention, the hydration activity of the parent nitrilase to 2-chloronicotinonitrile is eliminated, a byproduct of 2-chloronicotinamide is not generated in the catalytic process, the nitrile hydrolysis activity is greatly improved, and 2-chloronicotinonitrile can be specifically subjected to catalytic hydrolysis to synthesize 2-chloronicotinic acid. Therefore, the nitrilase mutant has an important potential in enzymatic industrial synthesis of 2-chloronicotinic acid.

### BRIEF DESCRIPTION OF THE DRAWINGS

FIG. 1 is an HPLC analysis spectrum of a reaction product of catalysis of 2-chloronicotinonitrile by a parent nitrilase (A) and a mutant W167G (B); and

FIG. 2 shows the progress of the hydrolysis reaction of 300 mM of 2-chloronicotinonitrile catalyzed by the mutant W167G.

### DETAILED DESCRIPTION OF THE EMBODIMENTS

The present invention is further described below in conjunction with the specific examples, but the protection scope of the present invention is not limited thereto.

### Example 1 Construction of Nitrilase Mutant Library

A pET-RZ plasmid containing a nitrilase gene (nucleotide sequence SEQ ID NO.1 and amino acid sequence SEQ ID NO.2) from Rhodococcus zopfii was used as a template, primers T7 F and T7 R (Table 1) were used for PCR amplification, and the mutation was introduced randomly.

PCR reaction system (50 μL): 0.5-20 ng of template pET-RZ, 1×Taq Buffer (without Mg²⁺), 0.2 mM of dNTP, 0.3 mM of MnCl₂, 2 mM of MgCl₂, 0.2 μM of each of upstream and downstream primers T7 F and T7 R, and 5 U of a Taq DNA polymerase.

PCR conditions: (1) pre-denaturation at 95° C. for 5 min; (2) denaturation at 95° C. for 15 s; (3) annealing at 60° C. for 5 s; (4) extension at 72° C. for 30 s, and a total of 30 cycles of steps (2)-(4); and (5) finally, extension at 72° C. for 3 min and preservation at 4° C.

A PCR product was analyzed by an agarose gel electrophoresis and a gel was cut for recovery.

Subsequently, the gel recovery product was used as primers for amplification to obtain complete a plasmid.

PCR system (50 μL): 2×Phanta Max buffer, 0.2 mM of dNTPs, 2.5 U of a Phanta Max high-fidelity polymerase, 50 ng of the gel recovery product, and 20 ng of the pET RZ plasmid.

PCR conditions: (1) pre-denaturation at 95° C. for 5 min; (2) denaturation at 95° C. for 15 seconds, annealing at 60° C. for 5 seconds, and extension at 72° C. for 3.5 min, and a total of 35 cycles of step (2); and (3) finally, extension at 72° C. for 5 min and preservation at 4° C.

The amplified PCR product was digested by an endonuclease DpnI at 37° C. for 3 hours, the enzyme was inactivated at 65° C. for 10 min, and the digested product was transformed into E. coli BL21 (DE3) which was coated on an LB plate containing kanamycin (50 μg/mL) and cultured overnight at 37° C.

TABLE 1
Primer design

| | Sequence of primers |
|---|---|
| Name of primers | (5′ to 3′) |
| T7 F | TAATACGACTCACTATAGGG |
| | (SEQ ID NO: 5) |
| T7 R | TGCTAGTTATTGCTCAGCGG |
| | (SEQ ID NO: 6) |
| W167 F | GGCGCGCTGAACTGCNNKGA |
| | ACACTTCCAGACC |
| | (SEQ ID NO: 7) |
| W167 R | GGTCTGGAAGTGTTCMNNGC |
| | AGTTCAGCGCGCC |
| | (SEQ ID NO: 8) |

### Example 2 High-Throughput Screening of Mutants

A single colony in example 1 was picked and cultured in a 96-deep well plate, 1 mL of an LB culture medium (containing kanamycin at a final concentration of 50 μg/mL) was added to each well plate, the bacteria were cultured at 37° C. for 12 hours, 200 μL of a bacterial solution was transferred to 800 μL of a fresh LB medium (at final concentration of 50 μg/mL of kanamycin, 0.1 mM IPTG), and the bacteria were cultured at 28° C. for 18 hours. The bacterial cells in the 96-deep well plate were centrifuged for 30 min (3,000 rpm, 4° C.), the supernatant was discarded, the residue was washed with a NaH₂PO₄-Na₂HPO₄ buffer solution (200 mM, pH 7.0), and the bacterial cells were resuspended with 600 μL of the buffer solution. A substrate 2-chloronicotinonitrile (at a final concentration of 100 mM) was added into each well and reaction was performed at 30° C. for 12 hours. After the reaction, a reaction solution in the 96-deep well plate was centrifuged for 30 min (3,000 rpm, 4° C.), 20 μL of a supernatant was transferred to a 96-well micro-reaction plate where each well contains 150 μL of a mixed solution of phthalaldehyde and mercaptoethanol, and the plate was placed at 37° C. for insulation for 30 min. The fluorescence intensity was measured with a microplate reader (excitation wavelength of 412 nm and emission wavelength of 467 nm). The stronger fluorescence indicated the higher nitrile hydrolysis activity and produced more NH₃ and 2-chloronicotinic acid. After verification by a liquid chromatography, a sequence analysis was performed. A mobile phase of the liquid chromatography is acetonitrile:water:phosphoric acid=250:750:1, the flow rate is 1 mL/min, and the detection wavelength is 210 nm. A mutant W167A (Table 2) was obtained by screening, that is, the codon at position 167 was mutated from TGG to GCC.

### Example 3 Site-Directed Mutagenesis

A plasmid pET-W167A was used as a template for site-directed mutagenesis by full plasmid amplification.

PCR system (50 μL): 0.5-20 ng of a template pET-W167A, 10-15 pmol of each of primers W167 F and W167 R, 2×Phanta Max buffer, 0.2 mM of dNTP, and 2.5 U of Phanta Max high-fidelity polymerase.

PCR conditions: (1) pre-denaturation at 95° C. for 5 min; (2) denaturation at 95° C. for 15 s, annealing at 60° C. for 5 s, and extension at 72° C. for 3.5 min, and a total of 35 cycles of step (2); and (3) finally, extension at 72° C. for 5 min and preservation at 4° C.

The amplified PCR product was digested by an endonuclease DpnI at 37° C. for 3 h, the enzyme was inactivated at 65° C. for 10 min, and the digested product was transformed into E. coli BL21 (DE3) which was coated on an LB plate containing kanamycin (50 μg/mL) and cultured overnight at 37° C.

A total of 200-300 monoclones were generated by saturation mutagenesis at position 167, and after sequencing, all 20 kinds of natural amino acids were included.

After preliminary screening by the high-throughput screening method in example 2, secondary screening and verification were performed by using liquid chromatography. It was determined that the mutants without nitrile hydration activity and with improved nitrile hydrolysis activity were W167S (i.e. the codon at position 167 mutated from TGG to TCC), W167C (i.e. the codon at position 167 mutated from TGG to TGC), and W167G (i.e. the codon at position 167 mutated from TGG to GGC), as shown in Table 2.

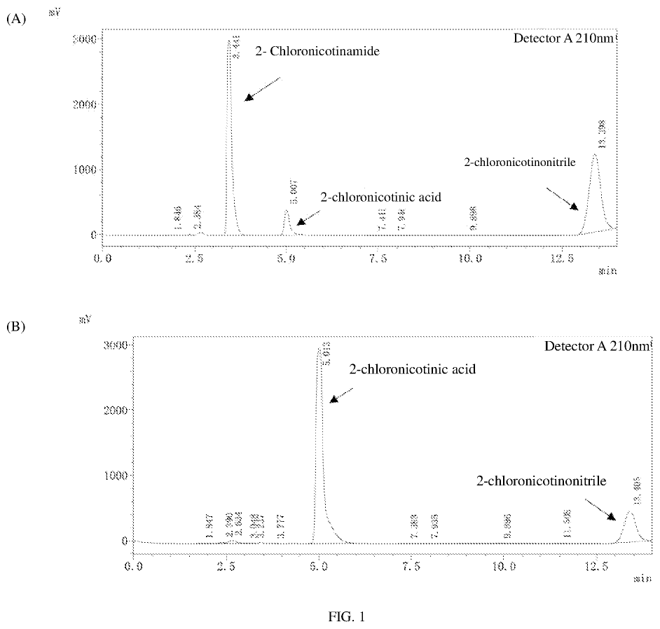

The parent nitrilase had the hydratase activity when hydrolyzing 2-chloronicotinonitrile, and produced a large amount of a byproduct of 2-chloronicotinamide (FIG. 1A). The modified mutant W167G only had the hydrolase activity, and did not produce the by-product of 2-chloronicotinamide (FIG. 1B).

TABLE 2
Comparison of relative activity of nitrilase

| Mutants | Proportion of amide (%) | Relative activity (%) |
|---|---|---|
| WT | 90.55 | 100 |
| W167A | 0.54 | 185 |
| W167S | 0 | 427 |
| W167C | 0 | 459 |
| W167G | 0 | 2052 |

### Example 4 Induction and Expression of Nitrilase

10 μL of the bacterial solution preserved in a glycerin tube was inoculated into 10 mL of a liquid LB medium (containing 50 μg/mL of kanamycin) and cultured at 37° C. and 200 rpm overnight, the cultured bacteria were transferred to 100 mL of a fresh LB medium (containing 50 μg/mL kanamycin) with an inoculum size of 2% and continuously cultured until OD₆₀₀ was 0.4-0.8, IPTG with a final concentration of 0.1 mM was added, and the bacteria were subjected to induced culture at 28° C. for 12 hours. After the culture, the bacterial cells were collected by centrifugation at 8,000 rpm and 4° C. for 10 min and washed twice with 0.9% normal saline to obtain wet bacterial cells.

### Example 5 Preparation of 2-Chloronicotinic Acid by Using Recombinant Nitrilase Mutant

The collected wet bacterial cells of the preferred mutant W167G in example 3 subjected to the induced culture by using the method in example 4 were used as a catalyst to be added into the reaction system (total system: 10 mL, and substrate: 300 mM of 2-chloronicotinonitrile, 200 mM of a NaH₂PO₄-Na₂HPO₄ buffer solution at a pH of 7, and 0.02 g of bacterial cells (dry weight)), and reaction was performed at 30° C. 100 μL of the reaction solution was sampled at regular intervals, 10 μL of 6 M HCl was added to terminate the reaction, and the content of the product was determined by HPLC.

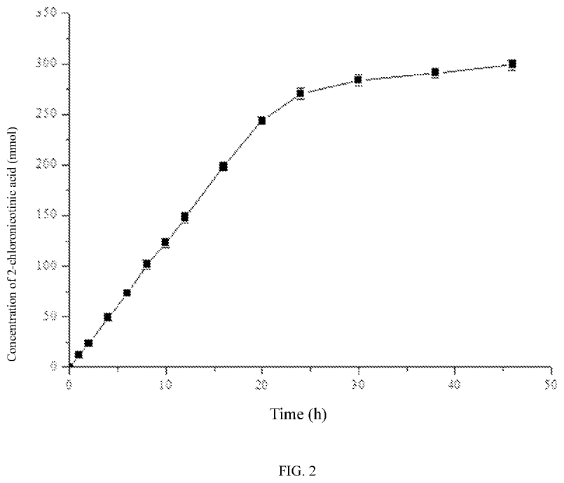

It can be seen from FIG. 2 that the mutant W167G can completely convert 300 mM of the substrate 2-chloronicotinonitrile into 2-chloronicotinic acid.

1. A nitrilase mutant having the amino acid sequence of SEQ ID NO.4.

2. A gene encoding the nitrilase mutant according to claim 1, wherein the nucleotide sequence of the gene is shown in SEQ ID No.3.

3. A recombinant plasmid comprising the gene according to claim 2.

4. The recombinant plasmid according to claim 3, wherein an original vector is pET-28b (+).

5. A recombinant engineered bacterium comprising the recombinant plasmid according to claim 3.

6. The recombinant engineered bacterium according to claim 5, wherein a host cell is Escherichia coli BL21.

7. A method of catalytic hydrolysis of 2-chloronicotinonitrile to synthesize 2-chloronicotinic acid comprising the step of utilizing the nitrilase mutant of claim 1.

8. The method according to claim 7, comprising subjecting a recombinant engineered bacterium comprising a gene encoding the nitrilase mutant to induced expression to obtain a wet bacterial cell, subjecting an immobilized cell of the wet bacterial cell or the wet bacterial cell to ultrasonication, extracting a pure enzyme as a catalyst, forming a reaction system by using 2-chloronicotinonitrile as a substrate and a NaH₂PO₄-Na₂HPO₄ buffer solution with a pH value of 6-8 as a reaction medium, performing reaction at 25-45° C., and performing separation and purification to obtain 2-chloronicotinic acid after the reaction.

9. The method according to claim 8, wherein in the reaction system, the amount of the catalyst is 0.2-3 g/L based on the dry weight of the bacterial cell, and the concentration of the substrate is 50-500 mM.

10. The method according to claim 9, wherein the bacterial cell collected after the induced culture is used as the catalyst, the NaH₂PO₄-Na₂HPO₄ buffer solution with a concentration of 200 mM and a pH value of 7 is used as the reaction medium, in the reaction system, the concentration of the substrate 2-chloronicotinonitrile is 300 mM, the bacterial cell is 2 g/L by dry weight, and the reaction is performed at 30° C. for 30-40 hours.

11. A recombinant engineered bacterium comprising the recombinant plasmid according to claim 4.


SEQUENCE LISTING (8 sequences)

SEQ ID NO: 1 - DNA, 1104 bp, Rhodococcus zopfii
atgggcgtggaatatactaataccttcaaagtagctgctgttcaggcacaacctgtgtggttcgatgcagcgaagaccgttgacaaaacggtatctatcatcgccgaggccgcacgtaacggttgcgagctggtggcattccctgaagtattcattccgggctacccgtaccacatctgggtcgactctccactggctggtatggccaaattcgctgtacgctaccacgaaaatagcctgactatggactccccgcacgtgcagcgtctgctggatgcggcccgtgaccacaatatcgcagttgttgttggtatttctgaacgtgatggtggttctctgtacatgactcagctggttattgacgcggatggtcaactggtggctcgtcgtcgcaaactgaaaccgactcacgtagaacgttctgtttacggtgaaggcaatggtagcgatatcagcgtttatgatatgccattcgctcgtctgggcgcgctgaactgctgggaacacttccagaccctgactaaatacgcgatgtatagcatgcacgaacaggttcacgtggcgagctggccgggtatgtctctgtaccagccggaggtaccggcatttggcgtggacgctcagctgaccgctacccgtatgtacgcgctggaaggtcaaaccttcgttgtttgtaccacccaagttgttactccggaagcccatgaattcttttgcgataacgatgaacagcgcaaactgatcggccgtggcggcggtttcgctcgtatcattggcccagacggtcgtgacctggcaacgccgctggccgaagacgaagagggtatcctgtatgcagacatcgacctgagcgctattaccctggcaaaacaggccgccgacccggtcggtcactactctcgcccggacgttctgtctctgaactttaaccagcgtcacaccaccccagtaaataccgcaatttctaccattcatgctacccacactctggtccctcagtctggcgctctggacggtgtacgcgagctgaacggcgcggacgagcagcgtgcgctgccgtccactcacagcgacgagactgatcgtgctactgcctctatctaa

SEQ ID NO: 2 - protein, 367 aa, Rhodococcus zopfii
MGVEYTNTFKVAAVQAQPVWFDAAKTVDKTVSIIAEAARNGCELVAFPEVFIPGYPYHIWVDSPLAGMAKFAVRYHENSLTMDSPHVQRLLDAARDHNIAVVVGISERDGGSLYMTQLVIDADGQLVARRRKLKPTHVERSVYGEGNGSDISVYDMPFARLGALNCWEHFQTLTKYAMYSMHEQVHVASWPGMSLYQPEVPAFGVDAQLTATRMYALEGQTFVVCTTQVVTPEAHEFFCDNDEQRKLIGRGGGFARIIGPDGRDLATPLAEDEEGILYADIDLSAITLAKQAADPVGHYSRPDVLSLNFNQRHTTPVNTAISTIHATHTLVPQSGALDGVRELNGADEQRALPSTHSDETDRATASI

SEQ ID NO: 3 - DNA, 1104 bp, Artificial Sequence
  Synthesized
atgggcgtggaatatactaataccttcaaagtagctgctgttcaggcacaacctgtgtggttcgatgcagcgaagaccgttgacaaaacggtatctatcatcgccgaggccgcacgtaacggttgcgagctggtggcattccctgaagtattcattccgggctacccgtaccacatctgggtcgactctccactggctggtatggccaaattcgctgtacgctaccacgaaaatagcctgactatggactccccgcacgtgcagcgtctgctggatgcggcccgtgaccacaatatcgcagttgttgttggtatttctgaacgtgatggtggttctctgtacatgactcagctggttattgacgcggatggtcaactggtggctcgtcgtcgcaaactgaaaccgactcacgtagaacgttctgtttacggtgaaggcaatggtagcgatatcagcgtttatgatatgccattcgctcgtctgggcgcgctgaactgcggcgaacacttccagaccctgactaaatacgcgatgtatagcatgcacgaacaggttcacgtggcgagctggccgggtatgtctctgtaccagccggaggtaccggcatttggcgtggacgctcagctgaccgctacccgtatgtacgcgctggaaggtcaaaccttcgttgtttgtaccacccaagttgttactccggaagcccatgaattcttttgcgataacgatgaacagcgcaaactgatcggccgtggcggcggtttcgctcgtatcattggcccagacggtcgtgacctggcaacgccgctggccgaagacgaagagggtatcctgtatgcagacatcgacctgagcgctattaccctggcaaaacaggccgccgacccggtcggtcactactctcgcccggacgttctgtctctgaactttaaccagcgtcacaccaccccagtaaataccgcaatttctaccattcatgctacccacactctggtccctcagtctggcgctctggacggtgtacgcgagctgaacggcgcggacgagcagcgtgcgctgccgtccactcacagcgacgagactgatcgtgctactgcctctatctaa

SEQ ID NO: 4 - protein, 367 aa, Artificial Sequence
  Synthesized
MGVEYTNTFKVAAVQAQPVWFDAAKTVDKTVSIIAEAARNGCELVAFPEVFIPGYPYHIWVDSPLAGMAKFAVRYHENSLTMDSPHVQRLLDAARDHNIAVVVGISERDGGSLYMTQLVIDADGQLVARRRKLKPTHVERSVYGEGNGSDISVYDMPFARLGALNCGEHFQTLTKYAMYSMHEQVHVASWPGMSLYQPEVPAFGVDAQLTATRMYALEGQTFVVCTTQVVTPEAHEFFCDNDEQRKLIGRGGGFARIIGPDGRDLATPLAEDEEGILYADIDLSAITLAKQAADPVGHYSRPDVLSLNFNQRHTTPVNTAISTIHATHTLVPQSGALDGVRELNGADEQRALPSTHSDETDRATASI

SEQ ID NO: 5 - DNA, 20 bp, Artificial Sequence
  Synthesized
taatacgactcactataggg

SEQ ID NO: 6 - DNA, 20 bp, Artificial Sequence
  Synthesized
tgctagttattgctcagcgg

SEQ ID NO: 7 - DNA, 33 bp, Artificial Sequence
  Synthesized
  misc_feature (16)..(17): n is a, c, g or t
ggcgcgctgaactgcnnkgaacacttccagacc

SEQ ID NO: 8 - DNA, 33 bp, Artificial Sequence
  Synthesized
  misc_feature (16)..(16): m is a, c, g or t
  misc_feature (17)..(18): n is a, c, g or t
ggtctggaagtgttcmnngcagttcagcgcgcc